(19) United States
(12) Patent Application Publication
(10) Pub. No.: US 2025/0360235 A1
(43) Pub. Date: Nov. 27, 2025

(54) RADIOLABELLING KIT AND METHOD FOR RADIOLABELLING

(71) Applicant: Telix Innovations SA, Herstal (BE)

(72) Inventors: Stephen THOMPSON, Herstal (BE); Eric WEBSTER, Herstal (BE); Jyoti ARORA, Herstal (BE)

(21) Appl. No.: 19/216,949

(22) Filed: May 23, 2025

Related U.S. Application Data
(60) Provisional application No. 63/650,984, filed on May 23, 2024.

(51) Int. Cl. A61K 51/04

(52) U.S. Cl. CPC A61K 51/0478; A61K 2123/00

(57) ABSTRACT

The present invention relates to methods and kits for radiolabelling a chelate-functionalized targeting agent with a metal radionuclide being gallium-68, using a stabiliser that prevents radiolysis (product degradation) of the chelate-functionalized targeting agent, wherein said stabiliser is selected from the group consisting of: ascorbic acid, dehydroascorbic acid, gentisic acid, cysteine and methionine, sodium ascorbate, or a salt thereof, preferably as a solution. The invention further relates to the use of said stabilizer against radiolytic decomposition of a radiolabelled chelate-functionalized targeting agent composition and methods of detecting a prostate tumour or cancer using the method for radiolabelling.

### CROSS-REFERENCE TO RELATED APPLICATIONS

This application claims the benefit of U.S. Provisional Application No. 63/650,984, filed on May 23, 2024, which is incorporated herein by reference in its entirety.

### TECHNICAL FIELD

The present invention is related to the field of nuclear medicine. More particular, the invention relates to radiolabelling of targeting agents, with radionuclides, more particular metal radionuclides. The obtained radiolabelled targeting agents may be used in therapeutic applications or in imaging techniques, such as Positron Emission Tomography (PET) and Single-Photon Emission Computed Tomography (SPECT), for example in vivo imaging of tumours and cancer, such as prostate cancer.

### BACKGROUND

As most radioisotopes used in nuclear medicine, and especially in the imaging applications, have a rather short half-life, the production of the radiolabel targeting agents is difficult to centralise, as the time to transport the radiolabelled targeting agents to the hospitals would take too long. Hence, most of the radiolabelled targeting agents are made on site, as also the radionuclides, especially metal radionuclides, can nowadays be generated on site by specifically therefore designed generators.

Therefore, kits and methods for production of such radiolabelled targeting agents on site, are in great demand. In recent years, the generators, but also cyclotrons, have been optimised and are able to produce higher radioactivity levels of radionuclides. This should allow to produce more patient doses in a single preparation method, or to store the prepared radiolabel targeting agents for a longer period of time before the radioactivity becomes too low for a patient dose.

However, when these higher radioactivity amounts are combined with the current kits and methods, the problem of radiolytic decomposition of the formed radiolabelled targeting agents become significant. Rapid disassociation of the radiolabelled targeting agent may occur under the influence of ionising radiation by the radionuclide, which in turn may drastically lower the performance of the targeting agent and generate impurities.

Therefore, even a small reduction in radiolytic decomposition of the formed radiolabelled targeting agents or optimalisation in radiolabelling targeting agents can have a large effect on purity and shelf-life of the radiolabelled targeting agents.

Hence, there is a need for radiolabelling kits and methods with high activity of radionuclides that avoid or reduce the radiolysis of the targeting agent during and/or after radiolabelling thereof, which are fast and easy to perform and do not require any complex lab equipment.

### SUMMARY OF THE INVENTION

In accordance with the purpose(s) of the disclosure, as embodied and broadly described herein, the disclosure, in one aspect, relates to radiolabelling of targeting agents with radionuclides. The disclosed radiolabelled targeting agents can be used in therapeutic applications or in imaging techniques, such as Positron Emission Tomography (PET) and Single-Photon Emission Computed Tomography (SPECT), e.g., for in vivo imaging of tumours and cancer, such as prostate cancer.

Disclosed are methods for radiolabelling a chelate-functionalized targeting agent with a metal radionuclide being gallium-68 or gallium-67, comprising the steps of: a) providing a stabiliser that prevents radiolysis (product degradation) of the chelate-functionalized targeting agent, wherein said stabiliser is selected from the group consisting of: ascorbic acid, dehydroascorbic acid, gentisic acid, cysteine and methionine, sodium ascorbate, or a salt thereof, preferably as a solution to the radiolabelling mixture prior to radiolabelling; b) providing a chelate-functionalized targeting agent, able to chelate the radioactive metal in the radiolabelling conditions; c) combining the mixture of a) and c); and, d) adding a radioactive metal to the mixture obtained in c), thereby radiolabelling the chelate-functionalized targeting agent with gallium-68 or gallium-67; wherein the method optionally further comprises mixing the stabiliser of a) with a buffering agent or buffer solution, allowing to maintain the pH in the range 3 to 8; and/or wherein the method optionally further comprises adding a metal inhibitor to said targeting agent of b), said metal inhibitor being a co-chelating agent, capable of inactivating metals other than radioactive metal without interfering with the chelation between the radioactive metal and the said chelate-functionalized targeting agent, under the conditions of the labelling reaction.

Also disclosed are compositions comprised a disclosed chelate-functionalized targeting agent comprising a radionuclide, e.g., gallium-68 or gallium-67.

Also disclosed compositions prepared by the disclosed methods and comprising a comprised a disclosed chelate-functionalized targeting agent comprising a radionuclide, e.g., gallium-68 or gallium-67.

Also disclosed are kits for producing a radiolabelled chelate-functionalized targeting agent with an activity of at least 50.0 mCi, comprising: (a) a chelate-functionalized targeting agent, able to chelate the radioactive metal in the radiolabelling conditions; (b) a stabiliser selected from the group consisting of: ascorbic acid, sodium ascorbate, dehydroascorbic acid, gentisic acid, cysteine and methionine, or a salt thereof, preferably as a solution; and (c) gallium-68 as radioactive metal; and, optionally one or more of: a metal inhibitor, which is a co-chelating agent, capable of inactivating metals other than radioactive metal without interfering with the chelation between the radioactive metal and the said chelate-functionalized targeting agent, under the conditions of the labelling reaction; and/or a buffering agent or buffer solution, allowing to maintain the pH in the range 3 to 8.

Also disclosed are methods of detecting a prostate tumour or cancer, comprising the steps of: (1) radiolabelling PSMA-11 (gozetotide) with gallium-68 according to the method claim 1; (2) administering to a subject a diagnostic amount of gallium-68 radiolabelled PSMA-11 (gozetotide); and, (3) detecting binding of said gallium-68 radiolabelled PSMA-11 (gozetotide) using PET or PET/CT imaging methods.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

In the following passages, different aspects or embodiments of the invention are defined in more detail. Every aspect or embodiment so defined may be combined with each of the other aspects or embodiments unless stated otherwise. In particular, any feature indicated as being preferred or advantageous in one embodiment may be combined with any other embodiment or embodiments indicated as being preferred or advantageous.

The present invention overcomes one or more of the problems identified and observed in the state of the art and allows stabilize radiolabelled chelate-functionalized targeting agents compositions with a high activity.

As used here, the term “stabilizer” refers to a compound with the ability to decrease or to prevent the radiolysis of the chelate-functionalized targeting agent and/or other compounds of the obtained radiolabelled chelate-functionalized targeting agent composition. Preferably, the stabilizer allows for a radiochemical purity of the radiolabelled chelate-functionalized targeting agent after 4 hours of at least 95%, and that preferably at radioactive concentrations higher than 7.0 mCi/ml. More preferably, the stabilizer allows for a radiochemical purity of the radiolabelled chelate-functionalized targeting agent after 6 hours of at least 95%, and that preferably at radioactive concentrations higher than 7.0 mCi/ml.

The stabiliser is preferably selected from the group consisting of: ascorbic acid, dehydroascorbic acid, gentisic acid, cysteine and methionine, or a salt thereof. A typical salt of ascorbic acid that can be used as stabilizer is sodium ascorbate. Alternative salts of ascorbic acid (so-called mineral ascorbates) could be calcium ascorbate, or magnesium ascorbate.

The present invention is related to the use of a stabiliser and optionally a metal inhibitor for improving shelf-life and radiolabelling yields of radioactive metal-based radiotracer synthesis, the radiolabelling being performed with:
A chelate-functionalized targeting agent, able to chelate the radioactive metal in the radiolabelling conditions;
A stabiliser that prevents radiolysis (product degradation) of the chelate-functionalized targeting agent, wherein said stabiliser is selected from the group consisting of: ascorbic acid, dehydroascorbic acid, gentisic acid, cysteine and methionine, sodium ascorbate, or a salt thereof, preferably as a solution;
A metal inhibitor, which is a co-chelating agent, capable of inactivating metals other than radioactive metal without interfering with the chelation between the radioactive metal and the said chelate-functionalized targeting agent, under the conditions of the labelling reaction. In other words, said metal inhibitor is selected for its ability to chelate contaminating metals interfering and competing with the chelation of the radioactive metal while being mostly unable to chelate the radioactive metal in the said conditions of the labelling reaction as opposed to the chelate-functionalized targeting agent;
A radioactive metal; and
Optionally, a buffering agent or buffer solution, allowing to maintain the pH in the range 3 to 8.

The present inventors have found that adding a certain amount of stabiliser to the radiolabelling solution prior to the radiolabelling reaction enables to avoid radiolysis of the chelate-functionalized targeting agent making it possible to use high activity radiolabelling conditions in the clinic. Through extensive experimentation, the inventors determined the optimal conditions needed for successfully avoiding radiolysis, thereby maintaining the radiochemical purity of the product and increasing the shelf-life of the kit provided herewith.

Furthermore, a metal inhibitor can be used in the radiolabelling method for neutralizing, at least partially, interfering species and allowing the radioactive metal to react with the chelate-functionalized targeting agent. These metal inhibitors may temporarily or permanently remove metals that compete with radioactive metal for the reaction with the chelate-functionalized targeting agent. Said metal inhibitor is thus unable to chelate the radioactive metal in the said conditions of the labelling reaction, but chelates other metals interfering with the chelation of radioactive metal by the chelate-functionalized targeting agent. The presence of metal inhibitors during the radiolabelling reaction provides an advantageous alternative to current approaches for managing the presence of metallic impurities such as increasing the amount of chelate-functionalized targeting agent or the pre-treatment of the eluate of the generator, these additional purification steps consume time (and radioactivity).

The aspects and embodiment as described herein advantageously allow to obtain an appropriate chelation yield, particularly above 95% up to even 100%, and therefore a very high radiochemical purity without any preliminary or further final purification and avoiding the need for heating. Said very high radiochemical purity can also be maintained over time when using high radioactivities of up to 500 mCi (from 10 mCi to 500 mCi).

As used herein, radioactivities are expressed in Curie [Ci] as unit. However, the conversion of Curie to Becquerel [Bq] is well known in the art, as 1 Ci=3.7·10¹⁰ Bq. Hence, 500 mCi is 1.85·10¹⁰ Bq.

The presence of a chelate-functionalized targeting agent, stabiliser, optionally a buffer and a metal inhibitor in the labelling medium advantageously allows to directly transfer the radioactive metal to the targeting agent and to perform the radiolabelling reaction without the need for any prior or subsequent operation or purification and avoiding the need for heating.

In some embodiments, all kit components as described herein can be lyophilized altogether or frozen which ensures a longer shelf life.

Thus, the main advantages of the invention as disclosed herein that differentiate from the state of the art are:
The possibility of radiolabelling without the need for an automated synthesizer, thereby opening up the possibility of using said kit with different generators, covering both liquid and solid target generators;
The possibility of a radiolabelling without the need for heating;
The presence of a metal inhibitor which advantageously allows to use less chelate-functionalized targeting agent and allowing the implementation of more affordable radiopharmaceutical synthesis; and
The presence of a metal inhibitor which advantageously allows improving the radiolabelling yields and avoiding the need for any purification of the eluate prior to radiolabelling as well as avoiding the need for any purification after radiolabelling.

In some embodiments, metal inhibitors used in the present invention are selected for their ability to block the competing metals in the radiolabelling reaction without inhibiting the radioactive metal ions in their chelation reaction with the chelate-functionalized targeting agent. Indeed, these metal inhibitors should not interfere negatively on the main radiolabelling reaction or lead to the formation of secondary radiolabelled species. In other words metal inhibitors should have a limited or no capacity to complex radioactive metal in the conditions used for the radiolabelling reaction. Limited means at least 100 times less than the chelating agent used for the radiolabelling of the chelate-functionalized targeting agent.

It is interesting to note that the function of metal inhibitors in some embodiments of the present invention is the opposite of the function of the sequestering agents generally used in the prior art. Indeed, according to known methods, at the end of the labelling reaction, a sequestering agent having a particular affinity for e.g. the radioactive gallium may be added to chelate the unreacted portion of the isotope, whereas, according to the present invention an agent capable of reducing the competition of metallic impurities other than the radioactive metal is added at the beginning of the reaction.

As used herein, an “inhibitor of metal” refers to any molecule capable of interacting with, or competing metals, or the chelating moiety of the chelate-functionalized targeting agent or with radioactive metal directly, to inhibit wholly or partially the chelation the chelate-functionalized targeting agent said competing metals and/or promote the chelating of radioactive metal by said targeting agent.

Metal inhibitors are preferably selected from the group of sugars. Sugars used as agents metal inhibitors in the kit of the invention are generally oligosaccharides (up to 6 or 7 monomeric sugar units or monosaccharides) and for example can be monosaccharides, disaccharides, trisaccharides (e.g. raffinose), tetrasaccharides (e.g. stachyose), or derivatives of monosaccharides such as tetracetose, pentacetose, hexacetose, tetrose, pentose, hexose, D-mannose, D-fructose, and derivatives; and/or disaccharides and their derivatives such as maltose and its derivatives; and/or cyclic oligosaccharides such as cyclodextrins and derivatives thereof.

Preferably, the metal inhibitor is present in the kit as described herein in micromolar amounts, preferably in nanomolar quantities, preferably in an amount of less than 500 nanomolar, still more preferably in an amount less than 100 nanomoles. In a preferred embodiment, said metal inhibitor is present in an amount of from 20 to 40 wt. % or from 25 to 35 wt. % based on the total weight of the chelate-functionalized targeting agent and metal inhibitor.

The metal inhibitory agent is usually not bound to the chelate-functionalized targeting agent but may also be chemically bound to the chelate-functionalized targeting agent when the chemical bond is a labile (breakable) bond under the conditions of radiolabelling with the chelate-functionalized targeting agent being released in situ in the conditions of radiolabelling. In one embodiment, said metal inhibitory agent is not chemically bound to the chelate-functionalized targeting agent.

As used herein, a “chelate-functionalized targeting agent” refers to a targeting agent capable of being labelled with a radioisotope such as for example radioactive metal, by means of an chelation agent to which this targeting agent is bound.

Preferred chelation agents for functionalizing a targeting agent to be radiolabelled with radioactive metals are those which form stable complexes at least for a time sufficient for diagnostic investigations using radiolabelled targeting agents. Suitable chelating agents include aliphatic amines, linear or macrocyclic such as macrocyclic amines with tertiary amines. While these examples of suitable chelating agents are not limited, they preferably include HBED or HBED-CC, DFO, EDTA, 6SS, B6SS, PLED, TAME, and YM103; NTP (PRHP)₃, H₂dedpa, (4,6-MeO₂sal)₂-BAPEN, and citrate and derivatives thereof. In a preferred embodiment, the chelator is HBED or a derivative thereof such as HBED-CC.

The chelate-functionalized targeting agent can comprise as a targeting moiety a peptide, for example, a peptide comprising 2 to 20 amino acids, a urea-based peptidomimetic, a polypeptide, a protein, a vitamin, a saccharide, for example a monosaccharide or a polysaccharide, an antibody, nucleic acid, an aptamer, an antisense oligonucleotide, or an organic molecule. In a preferred embodiment, said targeting agent is urea-based peptidomimetic Glu-urea-Lys.

In a particularly preferred embodiment, said chelate-functionalized targeting agent can be an urea-based (di) peptide or peptidomimetic, in one example, said chelate-functionalized targeting agent is PSMA-11 (HBED-CC functionalised Glu-urea-Lys), e.g. Glu-urea-Lys-HBED-CC known as Gozetotide.

Chelate-functionalized targeting agent as described herein preferably have a capacity of biological targeting. Non-limiting examples of suitable targeting agents include molecules that target PSMA validated in prostate cancer; Fibroblast Activation Protein Inhibitor (FAPi); CAIX (carbonic anhydrase IX) a scientifically validated target in cell renal cell carcinoma (ccRCC); large amino acid transporter LAT1 and LAT2 receptors validated targets that are highly expressed in several solid tumours, including malignancies of the central nervous system (CNS); cluster of differentiation 66 (CD66) for bone marrow conditioning; PDGFRα7 validated in soft tissue sarcoma (STS); VEGF receptors, analogues of bombesin or GRP receptor targeting molecules; molecules targeting somatostatin receptors; RGD peptides; or molecules targeting αvβ3 and αvβ5; annexin V; or molecules targeting the apoptotic process; molecules targeting oestrogen receptors; biomolecules targeting plaques; molecules targeting CD20; etc. More generally, a list of targeting molecules, organic or not, functionalized by a chelating can be found in Velikyan et al., Theranostic 2014, Vol. 4, Issue 1, “Prospective of 68Ga-Radiopharmaceutical Development” or in Zhang et al. Sig Transduct Target Ther 2025, Vol. 10, Issue 1, “Radiopharmaceuticals and their applications in medicine”.

The term “radioactive metal” as used herein for radioactive labelling of the functionalised targeting agent(s) encompasses all radioactive metal ions suitable for use in medical imaging or radionuclides therapy for the detection of prostate cancer and compatible with the chelators listed above. The radioactive metal typically is a gallium metal based radioisotope or radionuclide such as: gallium-68, gallium-67, or gallium-66. These radionuclides can be issued from nuclear reactor sub-products, cyclotron or from their specific radionuclide generator.

After addition of the radioactive metal solution to the mixture of chelate-functionalized targeting agent, stabiliser and optionally the metal inhibitor, optionally containing a buffer, the solution obtained is left to the radiolabelling reaction for a short period of time, in particular between about 2 minutes and about 60 minutes, preferably from about 2 minutes to about 30 minutes, for example about 2 to 5, 2 to 10, or 2 to 15 minutes at room temperature.

The invention also discloses a radiolabelled targeting agent with radioactive metal, obtained by a method as described herein.

In a specific embodiment, the radiolabelling kit comprises the following components:
Sterile Vial 1 comprising the chelate-functionalized targeting agent and the metal inhibitor, preferably in a sterile (e.g. 10 mL) vial;
Sterile Vial 2 that comprises buffering agent, preferably in a sterile (e.g. 10 mL) vial;
Vial 3 comprising an aqueous solution of radiolysis stabiliser selected from ascorbic acid, dehydroascorbic acid, gentisic acid, cysteine and methionine, sodium ascorbate, or a salt thereof.

More preferably, said kit comprises:
Sterile Vial 1, which comprises Gozetotide (PSMA-11) and D-mannose as a lyophilized powder in a sterile vial 10 mL vial, more preferably in an amount of 20 to 30 μg, most preferably of about 25 μg of Gozetotide and from 5 to 15 μg, more preferably about 10 μg of D-mannose; and/or
Sterile Vial 2, which comprises from 100 to 200 mg, preferably about 150 mg anhydrous sodium acetate in from 0,150 to 0,350 M HCl solution (2.5 mL volume to 6.4 mL volume); and/or
Sterile Vial 3, which comprises from 200 to 300 mg/mL, preferably about 250 mg/ml of ascorbic acid solution, preferably in a glass vial in order to maintain shelf-life of the stabiliser.

For use with some cyclotrons (liquid or solid target) as source of gallium, or for EZAG gallium generators (TiO₂-based), Vial 2 typically requires a higher molarity of the HCl solution, such as from 0,250 to 0,350 M HCl, more preferably of about 0,280 to 0,310 M HCl, most preferably of about 0,292 M HCl.

For use some cyclotrons (liquid or solid target) as source of gallium, or with IRE GalliEo type gallium generators (TiO₂-based), Vial 2 typically requires a lower molarity of the HCL solution, such as from 0,150 to 0,200 M HCl, more preferably from 0,170 to 0, 180 M HCl, most preferably of about 0,175 M HCl.

In some embodiments of the kits described herein, the amount of ascorbic acid, dehydroascorbic acid or a salt thereof in the kit, is so that the weight ratio of the weight of stabiliser over the weight of the buffering agent in the kit, is at least 0.006, preferably at least 0.010, preferably at least 0.030, preferably at least 0.050, preferably at least 0.100, preferably at least 0.150, preferably at least 0.200, preferably at least 0.250, preferably at least 0.300. In a preferred embodiment, said stabiliser is ascorbic acid.

In some embodiments of the kits described herein, the amount of stabiliser in the kit, is such that the weight ratio of the weight of stabiliser over the weight of the metal inhibitor in the kit, is weight of stabiliser over the weight of the metal inhibitor in the mixture obtained in step e), is at least 100, preferably at least 200, preferably at least 500, preferably at least 1000, preferably at least 2500, preferably at least 5000, preferably at least 10000, preferably at least 20000, preferably at least 50000. In a preferred embodiment, said stabiliser is ascorbic acid.

In some embodiments of the methods described herein, the amount of stabiliser in the mixture obtained in step e), is so that the weight ratio of the weight of stabiliser over the weight of the buffering agent in the mixture obtained in step e), is at least 0.006, preferably at least 0.010, preferably at least 0.030, preferably at least 0.050, preferably at least 0.100, preferably at least 0.150, preferably at least 0.200, preferably at least 0.250, preferably at least 0.300.

In some embodiments of the methods described herein, the amount of stabiliser in the mixture obtained in step e), is so that the weight ratio of the weight of stabiliser over the weight of the metal inhibitor in the mixture obtained in step e), is at least 100, preferably at least 200, preferably at least 500, preferably at least 1000, preferably at least 2500, preferably at least 5000, preferably at least 10000, preferably at least 20000, preferably at least 50000.

In some embodiments of the use described herein, the amount of stabiliser in the radiolabelled chelate-functionalized targeting agent composition, is so that the weight ratio of the weight of stabiliser over the weight of the buffering agent in the radiolabelled chelate-functionalized targeting agent composition, is at least 0.006, preferably at least 0.010, preferably at least 0.030, preferably at least 0.050, preferably at least 0.100, preferably at least 0.150, preferably at least 0.200, preferably at least 0.250, preferably at least 0.300. In a preferred embodiment, said stabiliser is ascorbic acid.

In some embodiments of the use described herein, the amount of stabiliser in the radiolabelled chelate-functionalized targeting agent composition, is so that the weight ratio of the equivalent weight of stabiliser over the weight of the metal inhibitor in the radiolabelled chelate-functionalized targeting agent composition, is at least 100, preferably at least 200, preferably at least 500, preferably at least 1000, preferably at least 2500, preferably at least 5000, preferably at least 10000, preferably at least 20000, preferably at least 50000. In a preferred embodiment, said stabiliser is ascorbic acid.

### Aspects

The following listing of exemplary aspects supports and is supported by the disclosure provided herein.
Aspect 1. A method for radiolabelling a chelate-functionalized targeting agent with a metal radionuclide being gallium-68 or gallium-67, comprising the steps of:
   a) providing a stabiliser that prevents radiolysis (product degradation) of the chelate-functionalized targeting agent, wherein said stabiliser is selected from the group consisting of: ascorbic acid, dehydroascorbic acid, gentisic acid, cysteine and methionine, sodium ascorbate, or a salt thereof, preferably as a solution to the radiolabelling mixture prior to radiolabelling;
   b) optionally, mixing the stabiliser of a) with a buffering agent or buffer solution, allowing to maintain the pH in the range 3 to 8;
   c) providing a chelate-functionalized targeting agent, able to chelate the radioactive metal in the radiolabelling conditions;
   d) optionally, adding a metal inhibitor to said targeting agent of c), said metal inhibitor being a co-chelating agent, capable of inactivating metals other than radioactive metal without interfering with the chelation between the radioactive metal and the said chelate-functionalized targeting agent, under the conditions of the labelling reaction;
   e) combining the mixture of a) and—when present—b), with the mixture of c) and—when present—d); and,
   f) adding a radioactive metal to the mixture obtained in e), thereby radiolabelling the chelate-functionalized targeting agent with gallium-68 or gallium-67.
Aspect 2. The method according to aspect 1, for radiolabelling a chelate-functionalized targeting agent with a metal radionuclide thereby producing a radiolabelled chelate-functionalized targeting agent with an activity of at least 50.0 mCi, preferably at least 60.0 mCi, preferably at least 70.0 mCi, preferably at least 80.0 mCi, preferably at least 90.0 mCi, preferably at least 100.0 mCi, preferably at least 150.0 mCi, preferably at least 200.0 mCi, preferably at least 250.0 mCi, preferably at least 300.0 mCi, preferably at least 350.0 mCi, preferably at least 400.0 mCi, preferably at least 450.0 mCi, preferably at least 500.0 mCi, preferably at least 750.0 mCi, preferably at least 1000.0 mCi.
Aspect 3. The method according to aspect 1 or 2, wherein the radioactive metal is provided as a solution with an radioactive concentration of at least 5.0 mCi/ml, preferably at least 6.0 mCi/ml, preferably at least 7.0 mCi/ml, preferably at least 10.0 mCi/ml, preferably at least 15.0 mCi/ml, preferably at least 20.0 mCi/ml, preferably at least 25.0 mCi/ml, preferably at least 30.0 mCi/ml, preferably at least 35.0 mCi/ml, preferably at least 40.0 mCi/ml, preferably at least 45.0 mCi/ml, preferably at least 50.0 mCi/ml, preferably at least 75.0 mCi/ml, preferably at least 100.0 mCi/ml.
Aspect 4. The method according to any one of previous aspects, wherein the mixture obtained in step e) comprises at least 10.0 mg, preferably at least 15.0 mg, preferably at least 20.0 mg, preferably at least 25.0 mg, preferably at least 30.0 mg of stabiliser, more preferably of ascorbic acid or a salt thereof such as sodium ascorbate.
Aspect 5. The method according to any one of previous aspects, wherein the mixture obtained in step e) comprises at most 100.0 mg, preferably at most 90.0 mg, preferably at most 80.0 mg, preferably at most 70.0 mg, preferably at most 60.0 mg, preferably at most 50.0 mg of stabiliser, more preferably of ascorbic acid or a salt thereof such as sodium ascorbate.
Aspect 6. The method according to any one of previous aspects, wherein the mixture obtained in step e) comprises at least 1.0 mg, preferably at least 1.2 mg, preferably at least 1.4 mg, preferably at least 1.6 mg, preferably at least 1.8 mg, preferably at least 2.0 mg, preferably at least 2.2 mg, preferably at least 2.4 mg, preferably at least 2.5 mg of stabiliser per 50 mCi metal radionuclide in step f);
more preferably wherein the mixture obtained in step e) comprises of at least 1.0 mg, preferably at least 1.2 mg, preferably at least 1.4 mg, preferably at least 1.6 mg, preferably at least 1.8 mg, preferably at least 2.0 mg, preferably at least 2.2 mg, preferably at least 2.4 mg, preferably at least 2.5 mg of ascorbic acid (or a salt thereof such as sodium ascorbate) per 50 mCi gallium-68 in step f).
Aspect 7. The method according to any one of previous aspects, wherein the mixture obtained in step e) comprises at most 100.0 mg, preferably at most 80.0 mg, preferably at most 60.0 mg, preferably at most 40.0 mg, preferably at most 20.0 mg, preferably at most 15.0 mg, preferably at most 10.0 mg, preferably at least 7.5 mg, preferably at most 5.0 mg of stabiliser per 50 mCi metal radionuclide in step f);
more preferably wherein the mixture obtained in step e) comprises of at most 100.0 mg, preferably at most 80.0 mg, preferably at most 60.0 mg, preferably at most 40.0 mg, preferably at most 20.0 mg, preferably at most 15.0 mg, preferably at most 10.0 mg, preferably at least 7.5 mg, preferably at most 5.0 mg of ascorbic acid (or a salt thereof such as sodium ascorbate) per 50 mCi gallium-68 in step f).
Aspect 8. The method according to any one of previous aspects, wherein the amount of stabiliser in the mixture obtained in step e), is so that the weight ratio of the weight of stabiliser over the weight of the chelate-functionalized targeting agent in the mixture obtained in step e), is at least 40, preferably at least 80, preferably at least 150, preferably at least 300, preferably at least 600, preferably at least 1000, preferably at least 1500, preferably at least 2000.
Aspect 9. The method according to any one of previous aspects, wherein the solution of stabiliser, has a concentration of at least 0.14 mg/ml, preferably at least 0.30 mg/ml, preferably at least 0.50 mg/ml, preferably at least 1.00 mg/ml, preferably at least 2.00 mg/ml, preferably at least 4.00 mg/ml, preferably at least 5.00 mg/ml, preferably at least 7.00 mg/ml, preferably at least 9.00 mg/ml, preferably at least 10.00 mg/ml, preferably wherein the stabilizer is ascorbic acid or a salt thereof such as sodium ascorbate.
Aspect 10. The method according to any one of previous aspects, wherein the stabiliser is ascorbic acid, dehydroascorbic acid or a salt thereof, and optionally wherein said stabiliser is provided together with a bisulfite and or a metabisulfite.
Aspect 11. The method according to any one of previous aspects, for providing at least 3.0, preferably at least 4.0, preferably at least 5.0, preferably at least 6.0, preferably at least 8.0, preferably at least 10.0, preferably at least 12.0, preferably at least 15.0, preferably at least 20.0 patient doses.
Aspect 12. The method according to any one of previous aspects, wherein said targeting agent and metal inhibitor are present in a buffer allowing to maintain the pH in the range 3 to 8.
Aspect 13. The method according to any one of previous aspects, wherein said targeting agent and metal inhibitor are present in a buffer selected from the group consisting of: phosphate, nitrate, HEPES, acetate, formate, TRIS, and citrate or a mixture thereof, preferably in an acetate buffer, more preferably a sodium acetate buffer.
Aspect 14. The method according to any one of previous aspects, wherein the chelate functional group of the targeting agent is HBED or derivatives thereof such as HBED-CC.
Aspect 15. The method according to any one of previous aspects, wherein said metal inhibitor is a sugar, preferably a short-chain sugar or oligosaccharide, such as comprising up to 7 monosaccharide units.
Aspect 16. The method according to any one of previous aspects, wherein said metal inhibitor is selected from the group comprising: monosaccharides and their derivatives, disaccharides and their derivatives, trisaccharides such as raffinose, tetrasaccharides such as stachyose, and cyclic oligosaccharides such as cyclodextrins.
Aspect 17. The method according to any one of previous aspects, wherein said metal inhibitor is selected from the group comprising: Glucose, D-Fructose, Beta-cyclodextrin, and D-Mannose, more preferably D-mannose.
Aspect 18. The method according to any one of previous aspects, wherein said metal inhibitor and said functionalised agent are not chemically linked.
Aspect 19. The method according to any one of previous aspects, wherein said metal inhibitor and said functionalised agent are chemically linked, through a linker that is unstable in the radiolabelling conditions.
Aspect 20. The method according to any one of previous aspects, wherein said chelate-functionalized targeting agent is Glu-urea-Lys-HBED-CC (gozetotide or PSMA-11).
Aspect 21. The method according to any one of previous aspects, wherein the radiolabelling reaction is carried out at ambient or room temperature (such as between 15 and 30° C.).
Aspect 22. The method according to any one of previous aspects, wherein the radiolabelling is performed at a pH comprised between 3 and 8, preferably between 3.5 and 7.5, more preferably between 3.5 and 7.
Aspect 23. A radiolabelled chelate-functionalized targeting agent obtained by the method according to anyone of aspects 1 to 22.
Aspect 24. The radiolabelled chelate-functionalized targeting agent according to aspect 23, which is gallium-68 radiolabelled Glu-urea-Lys-HBED-CC (gozetotide or PSMA-11).
Aspect 25. A radiolabelling kit, preferably suitable for producing a radiolabelled chelate-functionalized targeting agent with an activity of at least 50.0 mCi, comprising:
   a chelate-functionalized targeting agent, able to chelate the radioactive metal in the radiolabelling conditions;
   optionally, a metal inhibitor, which is a co-chelating agent, capable of inactivating metals other than radioactive metal without interfering with the chelation between the radioactive metal and the said chelate-functionalized targeting agent, under the conditions of the labelling reaction;
   a stabiliser selected from the group consisting of: ascorbic acid, sodium ascorbate, dehydroascorbic acid, gentisic acid, cysteine and methionine, or a salt thereof, preferably as a solution;
   gallium-68 as radioactive metal; and,
   optionally, a buffering agent or buffer solution, allowing to maintain the pH in the range 3 to 8.
Aspect 26. The radiolabelling kit according to aspect 25, wherein the chelate-functionalized targeting agent and the metal inhibitor are provided in an lyophilized form, preferably together in one vial.
Aspect 27. The kit according to aspect 25 or 26, suitable for radiolabelling a chelate-functionalized targeting agent with gallium-68 thereby producing a gallium-68 chelate-functionalized targeting agent with an activity of at least 50.0 mCi, preferably at least 100.0 mCi, preferably at least 150.0 mCi, preferably at least 200.0 mCi, preferably at least 250.0 mCi, preferably at least 300.0 mCi, preferably at least 350.0 mCi, preferably at least 400.0 mCi, preferably at least 450.0 mCi, preferably at least 500.0 mCi, preferably at least 750.0 mCi, preferably at least 1000.0 mCi.
Aspect 28. The kit according to any one of aspects 25 to 27, wherein the gallium-68 is provided as a solution with an activity of at least 5.0 mCi/ml, preferably at least 7.0 mCi/ml, preferably at least 10.0 mCi/ml, preferably at least 15.0 mCi/ml, preferably at least 20.0 mCi/ml, preferably at least 25.0 mCi/ml, preferably at least 30.0 mCi/ml, preferably at least 35.0 mCi/ml, preferably at least 40.0 mCi/ml, preferably at least 45.0 mCi/ml, preferably at least 50.0 mCi/ml, preferably at least 75.0 mCi/ml, preferably at least 100.0 mCi/ml.
Aspect 29. The kit according to any one of aspects 25 to 28, comprising at least 10.0 mg, preferably at least 15.0 mg, preferably at least 20.0 mg, preferably at least 25.0 mg, preferably at least 30.0 mg of stabiliser, wherein said stabiliser preferably is ascorbic acid or a salt thereof such as sodium ascorbate.
Aspect 30. The kit according to any one of aspects 25 to 29, wherein the kit comprises at most 100.0 mg, preferably at most 90.0 mg, preferably at most 80.0 mg, preferably at most 70.0 mg, preferably at most 60.0 mg, preferably at most 50.0 mg of stabiliser, more preferably of ascorbic acid or a salt thereof such as sodium ascorbate.
Aspect 31. The kit according to any one of aspects 25 to 30, wherein the kit comprises at least 1.0 mg, preferably at least 1.2 mg, preferably at least 1.4 mg, preferably at least 1.6 mg, preferably at least 1.8 mg, preferably at least 2.0 mg, preferably at least 2.2 mg, preferably at least 2.4 mg, preferably at least 2.5 mg of stabiliser per 50 mCi metal radionuclide;
more preferably wherein the kit comprises of at least 1.0 mg, preferably at least 1.2 mg, preferably at least 1.4 mg, preferably at least 1.6 mg, preferably at least 1.8 mg, preferably at least 2.0 mg, preferably at least 2.2 mg, preferably at least 2.4 mg, preferably at least 2.5 mg of ascorbic acid (or a salt thereof such as sodium ascorbate) per 50 mCi gallium-68.
Aspect 32. The kit according to any one of aspects 25 to 31, wherein the kit comprises at most 100.0 mg, preferably at most 80.0 mg, preferably at most 60.0 mg, preferably at most 40.0 mg, preferably at most 20.0 mg, preferably at most 15.0 mg, preferably at most 10.0 mg, preferably at least 7.5 mg, preferably at most 5.0 mg of stabiliser per 50 mCi metal radionuclide;
more preferably wherein the kit comprises of at most 100.0 mg, preferably at most 80.0 mg, preferably at most 60.0 mg, preferably at most 40.0 mg, preferably at most 20.0 mg, preferably at most 15.0 mg, preferably at most 10.0 mg, preferably at least 7.5 mg, preferably at most 5.0 mg of ascorbic acid (or a salt thereof such as sodium ascorbate) per 50 mCi gallium-68.
Aspect 33. The kit according to any one of aspects 25 to 32, comprising at least 10.0 μg, preferably at least 15.0 μg, preferably at least 20.0 μg, preferably at least 25.0 μg of said chelate-functionalized targeting agent.
Aspect 34. The kit according to any one of aspects 25 to 33, comprising at least 2.0 μg, preferably at least 4.0 μg, preferably at least 6.0 μg, preferably at least 8.0 μg, preferably at least 10.0 μg of said metal inhibitor.
Aspect 35. The kit according to any one of aspects 25 to 34, comprising at least 50.0 mg, preferably at least 75.0 mg, preferably at least 100.0 mg, preferably at least 125.0 mg, preferably at least 150.0 mg of said buffering agent.
Aspect 36. The kit according to any one of aspects 25 to 35, wherein the amount of stabiliser in the kit, is such that the weight ratio of the weight of stabiliser over the weight of the chelate-functionalized targeting agent in the kit, is at least 40, preferably at least 80, preferably at least 150, preferably at least 300, preferably at least 600, preferably at least 1000, preferably at least 1500, preferably at least 2000. In a preferred embodiment, said stabiliser is ascorbic acid.
Aspect 37. The kit according to any one of aspects 25 to 36, wherein the solution of stabiliser, has a concentration of at least 0.14 mg/ml, preferably at least 0.30 mg/ml, preferably at least 0.50 mg/ml, preferably at least 1.00 mg/ml, preferably at least 2.00 mg/ml, preferably at least 4.00 mg/ml, preferably at least 5.00 mg/ml, preferably at least 7.00 mg/ml, preferably at least 9.00 mg/ml, preferably at least 10.00 mg/ml. In a preferred embodiment, said stabiliser is ascorbic acid.
Aspect 38. The kit according to any one of aspects 25 to 37, wherein the stabiliser is provided in the kit together with a bisulfite and or a metabisulfite.
Aspect 39. The kit according to any one of aspects 25 to 38, wherein the kit is suitable for providing at least 3.0, preferably at least 4.0, preferably at least 5.0, preferably at least 6.0, preferably at least 8.0, preferably at least 10.0, preferably at least 12.0, preferably at least 15.0, preferably at least 20.0 patient doses.
Aspect 40. The kit according to any one of aspects 25 to 39, wherein said targeting agent and metal inhibitor are present in a buffer selected from the group consisting of: phosphate, nitrate, HEPES, acetate, formate, TRIS, and citrate or a mixture thereof, preferably an acetate buffer, more preferably a sodium acetate buffer.
Aspect 41. The kit according to any one of aspects 25 to 40, wherein the chelate functional group of the targeting agent is HBED or derivatives thereof such as HBED-CC.
Aspect 42. The kit according to any one of aspects 25 to 41, wherein said metal inhibitor is a sugar, preferably a short-chain sugar or oligosaccharide, such as comprising up to 7 monosaccharide units.
Aspect 43. The kit according to anyone of aspects 25 to 42, wherein said metal inhibitor is selected from the group comprising: monosaccharides and their derivatives, disaccharides and their derivatives, trisaccharides such as raffinose, tetrasaccharides such as stachyose, and cyclic oligosaccharides such as cyclodextrins.
Aspect 44. The kit according to anyone of aspects 25 to 43, wherein said metal inhibitor is selected from the group comprising: Glucose, D-Fructose, Beta-cyclodextrin, and D-Mannose.
Aspect 45. The kit according to any one of aspects 25 to 44, wherein said metal inhibitor and said functionalised agent are not chemically linked.
Aspect 46. The kit according to any one of aspects 25 to 45, wherein said metal inhibitor and said functionalised agent are chemically linked, through a linker that is unstable in the radiolabelling conditions.
Aspect 47. The kit according to any one of aspects 25 to 46, wherein said chelate-functionalized targeting agent is Glu-urea-Lys-HBED-CC (gozetotide or PSMA-11).
Aspect 48. The kit according to any one of aspects 25 to 47, wherein said chelate-functionalized targeting agent and metal inhibitor are lyophilised.
Aspect 49. Use of a stabiliser selected from the group consisting of: ascorbic acid, dehydroascorbic acid, gentisic acid, cysteine and methionine, sodium ascorbate, or a salt thereof against radiolytic decomposition of a radiolabelled chelate-functionalized targeting agent composition with an activity of at least 50 mCi, preferably at least 100.0 mCi, preferably at least 150.0 mCi, preferably at least 200.0 mCi, preferably at least 250.0 mCi, preferably at least 300.0 mCi, preferably at least 350.0 mCi, preferably at least 400.0 mCi, preferably at least 450.0 mCi, preferably at least 500.0 mCi, preferably at least 750.0 mCi, preferably at least 1000.0 mCi.
Aspect 50. The use according to aspect 49, wherein said stabiliser is used in an amount equivalent to at least 10.0 mg, preferably at least 15.0 mg, preferably at least 20.0 mg, preferably at least 25.0 mg, preferably at least 30.0 mg stabiliser. In a preferred embodiment, said stabiliser is ascorbic acid.
Aspect 51. The use according to aspect 49 or 50, wherein said stabiliser is used in an amount equivalent to at most 100.0 mg, preferably at most 90.0 mg, preferably at most 80.0 mg, preferably at most 70.0 mg, preferably at most 60.0 mg, preferably at most 50.0 mg of stabiliser, more preferably of ascorbic acid or a salt thereof such as sodium ascorbate.
Aspect 52. The use according to any one of aspects 49 to 51, wherein said stabiliser is used in an amount equivalent to at least 1.0 mg, preferably at least 1.2 mg, preferably at least 1.4 mg, preferably at least 1.6 mg, preferably at least 1.8 mg, preferably at least 2.0 mg, preferably at least 2.2 mg, preferably at least 2.4 mg, preferably at least 2.5 mg of stabiliser per 50 mCi metal; more preferably wherein said stabiliser is used in an amount equivalent to at least 1.0 mg, preferably at least 1.2 mg, preferably at least 1.4 mg, preferably at least 1.6 mg, preferably at least 1.8 mg, preferably at least 2.0 mg, preferably at least 2.2 mg, preferably at least 2.4 mg, preferably at least 2.5 mg of ascorbic acid (or a salt thereof such as sodium ascorbate) per 50 mCi gallium-68.
Aspect 53. The use according to any one of aspects 49 to 52, wherein said stabiliser is used in an amount equivalent to at most 100.0 mg, preferably at most 80.0 mg, preferably at most 60.0 mg, preferably at most 40.0 mg, preferably at most 20.0 mg, preferably at most 15.0 mg, preferably at most 10.0 mg, preferably at least 7.5 mg, preferably at most 5.0 mg of stabiliser per 50 mCi metal radionuclide;
more preferably wherein said stabiliser is used in an amount equivalent to at most 100.0 mg, preferably at most 80.0 mg, preferably at most 60.0 mg, preferably at most 40.0 mg, preferably at most 20.0 mg, preferably at most 15.0 mg, preferably at most 10.0 mg, preferably at least 7.5 mg, preferably at most 5.0 mg of ascorbic acid (or a salt thereof such as sodium ascorbate) per 50 mCi gallium-68.
Aspect 54. The use according to any one of aspects 49 to 53, wherein the amount of stabiliser in the radiolabelled chelate-functionalized targeting agent composition, is so that the weight ratio of the weight of stabiliser over the weight of the chelate-functionalized targeting agent in the radiolabelled chelate-functionalized targeting agent composition, is at least 40, preferably at least 80, preferably at least 150, preferably at least 300, preferably at least 600, preferably at least 1000, preferably at least 1500, preferably at least 2000. In a preferred embodiment, said stabiliser is ascorbic acid or a salt thereof such as sodium ascorbate.
Aspect 55. The use according to any one of aspects 49 to 54, wherein the stabiliser is used as a solution and has a concentration of at least 0.14 mg/ml, preferably at least 0.30 mg/ml, preferably at least 0.50 mg/ml, preferably at least 1.00 mg/ml, preferably at least 2.00 mg/ml, preferably at least 4.00 mg/ml, preferably at least 5.00 mg/ml, preferably at least 7.00 mg/ml, preferably at least 9.00 mg/ml, preferably at least 10.00 mg/ml. In a preferred embodiment, said stabiliser is ascorbic acid or a salt thereof such as sodium ascorbate.
Aspect 56. The use according to any one of aspects 49 to 55, wherein the stabiliser is used together with a bisulfite and or a metabisulfite.
Aspect 57. The use according to any one of aspects 49 to 56, for the stabilization of at least 3.0, preferably at least 4.0, preferably at least 5.0, preferably at least 6.0, preferably at least 8.0, preferably at least 10.0, preferably at least 12.0, preferably at least 15.0, preferably at least 20.0 patient doses.
Aspect 58. The use according to any one of aspects 49 to 57, wherein the radiolabelled chelate-functionalized targeting agent composition comprises a buffer allowing to maintain the pH in the range 3 to 8.
Aspect 59. The use according to any one of aspects 49 to 58, wherein the buffer is selected from the group consisting of: phosphate, nitrate, HEPES, acetate, formate, TRIS, and citrate or a mixture thereof, preferably wherein said buffer comprises acetate, more preferably a sodium acetate buffer.
Aspect 60. The use according to any one of aspects 49 to 59, wherein the chelate functional group of the targeting agent is HBED or derivatives thereof such as HBED-CC
Aspect 61. The use according to any one of aspects 49 to 60, wherein said metal inhibitor is a sugar, preferably a short-chain sugar or oligosaccharide, such as comprising up to 7 monosaccharide units.
Aspect 62. The use according to any one of aspects 49 to 61, wherein said metal inhibitor is selected from the group comprising: monosaccharides and their derivatives, disaccharides and their derivatives, trisaccharides such as raffinose, tetrasaccharides such as stachyose, and cyclic oligosaccharides such as cyclodextrins.
Aspect 63. The use according to any one of aspects 49 to 62, wherein said metal inhibitor is selected from the group comprising: Glucose, D-Fructose, Beta-cyclodextrin, and D-Mannose, more preferably D-mannose.
Aspect 64. The use according to any one of aspects 49 to 63, wherein said metal inhibitor and said functionalised agent are not chemically linked.
Aspect 65. The use according to any one of aspects 49 to 64, wherein said metal inhibitor and said functionalised agent are chemically linked, through a linker that is unstable in the radiolabelling conditions.
Aspect 66. The use according to any one of aspects 49 to 65, wherein said chelate-functionalized targeting agent is PSMA-11 (gozetotide).
Aspect 67. A method of detecting a prostate tumour or cancer, comprising the steps of:
   1) radiolabelling PSMA-11 (gozetotide) with gallium-68 according to the method of any one of aspects 1 to 22,
   2) administering to a subject a diagnostic amount of gallium-68 radiolabelled PSMA-11 (gozetotide); and,
   3) detecting binding of said gallium-68 radiolabelled PSMA-11 (gozetotide) using PET or PET/CT imaging methods.
Aspect 68. The method according to aspect 67, wherein said detection is used for:
   (i) initial staging of prostate cancer into intermediate, unfavourable, high, or very high risk prostate cancer,
   (ii) detecting suspected recurrence of prostate cancer and/or detection of metastasis,
   (iii) selection for radiotherapeutic treatment such as with Lutetium (177Lu) vipivotide tetraxetan (Pluvicto)
   (iv) monitoring prostate cancer for progression into Non-Metastatic or Metastatic Castration-Resistant Prostate Cancer (nmCRPC or mCRPC), or
   (v) determining response to (radio) therapy.
Aspect 69. The method according to aspect 67 or aspect 68, wherein said detection method is used to replace the need for taking a prostate biopsy or is used in PET or PET/CT scan with MRI in clinically significant or intermediate favourable prostate cancers, or in MRI for active surveillance of prostate cancer. From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

### Example 1

A kit comprising 3 vials, wherein:
the first vial comprises 25.0 μg PSMA-11, and 10.0 μg d-mannose as a lyophilised powder in a sterile 10 ml vial;
the second vial comprises 150.0 mg anhydrous sodium acetate in 2.5 ml 0.292 M HCl solution in a sterile 10 ml vial;
the third vial comprising at least 25.0 mg ascorbic acid, as a 250 mg/ml aqueous solution (as provided by Sandoz).

The kit further comprises the eluate of a EZAG GalliaPharm generator or the product of GE Liquid used with FastLab, typically comprising 60 mCi-70 mCi [⁶⁸Ga]GaCl₃, typically the volume of the eluate is about 5 ml.

### Radiolabelling Procedure

100 μl of the ascorbic acid solution (equivalent to 25 mg ascorbic acid) was taken from vial 3, and was filtered through a 5 μm filter needle. The sodium ascorbate solution was subsequently added to vail 2, and mixed thoroughly. The obtained content of vial 2 was added to vial 1, and again mixed thoroughly. To this obtained vail 1, the eluate, comprising the gallium-68, was added to obtain a gallium-68 labelled PSMA-11 solution. Optionally, a 2.5 ml 0.9% sodium chloride solution may be added to increase the volume and/or to amend the osmolality to be under 1000 mOsm/kg.

The pH of the gallium-68 labelled PSMA-11 solution was 4.5 immediately after the radiolabelling procedure and was unchanged after 4 hours.

### Patient Administration

The obtained Gallium-68 labelled PSMA-11 solution can be divided in patient doses comprising a radioactivity of 5 mCi±10%), and can be administered to a patient intravenously. The patient may be subjected to a positron emission tomography (PET) scan or PET/CT scan, to detect prostate-specific membrane antigen (PSMA) positive lesions in men with prostate cancer.

### Example 2

A kit comprising 3 vials, wherein:
the first vial comprises 25.0 μg PSMA-11, and 10.0 μg d-mannose as a lyophilised powder in a sterile 10 ml vial;
the second vial comprises 150.0 mg anhydrous sodium acetate in 6.4 ml 0.175 M HCl solution in a sterile 10 ml vial;
the third vial comprising at least 25.0 mg ascorbic acid, as a 250 mg/ml aqueous solution (as provided by Sandoz).

The kit further comprises the eluate of a IRE Galli Eo generator, typically comprising 60 mCi-70 mCi [⁶⁸Ga]GaCl₃, typically the volume of the eluate is about 1.1 ml.

### Radiolabelling Procedure

100 μl of the ascorbic acid solution (equivalent to 25 mg ascorbic acid) was taken from vial 3, and was filtered through a 5 μm filter needle. The sodium ascorbate solution was subsequently added to vail 2, and mixed thoroughly. The obtained content of vial 2 was added to vial 1, and again mixed thoroughly. To this obtained vail 1, the eluate, comprising the gallium-68, was added to obtain a Gallium-68 labelled PSMA-11 solution. Optionally, a sodium chloride solution may be added to increase the volume of the obtained Gallium-68 labelled PSMA-11 solution and/or to amend the osmolality to be under 1000 mOsm/kg. The pH of the gallium-68 labelled PSMA-11 solution was 4.5 immediately after the radiolabelling procedure and was unchanged after 4 hours.

### Patient Administration

The obtained Gallium-68 labelled PSMA-11 solution can be divided in patient doses comprising a specific activity of 5 mCi±10%), and can be administered to a patient intravenously. The patient may be subjected to a positron emission tomography (PET) scan or PET/CT scan, to detect prostate-specific membrane antigen (PSMA) positive lesions in men with prostate cancer.

### Example 3

A kit comprising 3 vials, wherein:
the first vial comprises 25.0 μg PSMA-11, and 10.0 μg d-mannose as a lyophilised powder in a sterile 10 ml vial;
the second vial comprises 150.0 mg anhydrous sodium acetate in 2.5 ml 0.292 M HCl solution in a sterile 10 ml vial;
the third vial comprising at least 25.0 mg ascorbic acid, as a 250 mg/ml aqueous solution (as provided by Sandoz).

The kit further comprises the eluate of:
a GE Solid Target used with FastLab; or,
an ARTMS Solid Target QIS system with Trasis AIO;
typically comprising 350 mCi-600 mCi [⁶⁸Ga]GaCl₃, typically the volume of the eluate is about 1.1 to 5 ml.

### Radiolabelling Procedure

100 μl of the ascorbic acid solution (equivalent to 25 mg ascorbic acid) was taken from vial 3, and was filtered through a 5 μm filter needle. The sodium ascorbate solution was subsequently added to vail 2, and mixed thoroughly. The obtained content of vial 2 was added to vial 1, and again mixed thoroughly. To this obtained vail 1, the eluate, comprising the gallium-68, was added to obtain a Gallium-68 labelled PSMA-11 solution. Optionally, a sodium chloride solution may be added to increase the volume of the obtained Gallium-68 labelled PSMA-11 solution and/or to amend the osmolality to be under 1000 mOsm/kg. The pH of the gallium-68 labelled PSMA-11 solution was 4.5 immediately after the radiolabelling procedure and was unchanged after 6 hours.

### Patient Administration

The obtained Gallium-68 labelled PSMA-11 solution was divided in patient doses comprising a specific activity of 5 mCi±10%), and administered to a patient intravenously. The patient may be subjected to a positron emission tomography (PET) scan or PET/CT scan, to detect prostate-specific membrane antigen (PSMA) positive lesions in men with prostate cancer.

### Example 4: Radiochemical Purity Results

Table 1 to Table 5 show the results of various radiolabelling experiments on radiochemical purity and the chelation yield. The radiolabelling experiments in the tables are carried out as described in:
Example 1 for when the gallium-68 source is GE Liquid target generator and EZAG GalliaPharm generator;
Example 2 for when the gallium-68 source is IRE Galli Eo generator;
Example 3 for when the gallium-68 source is GE solid target generator;

However, the experiments were carried out with the amount of ascorbic acid as indicated in the Tables 1 to 5, and the activity as indicated in the Tables 1 to 5. Radiochemical purities were measured at various times (T0, T1, T4, T6) after the radiolabelled experiments have been conducted.

TABLE 1
Radiochemical purity data for batches of [⁶⁸Ga]PSMA-11 prepared with <70
mCi of [⁶⁸Ga]GaCl₃ with the inclusion of 1 mg, 2.5 mg, 5 mg and 15 mg of ascorbic acid.

| Mass of Ascorbic Acid | | 1 mg | | 2.5 mg | | 5 mg | | 15 mg | | 15 mg | |
|---|---|---|---|---|---|---|---|---|---|---|---|
| Included in the final | | | | | | | | | | | |
| radiolabelled product | | | | | | | | | | | |
| Activity Added to TLX-007 | | 58.7 -71.1 | | 49.9-76.9 | | 60-69.9 | | 23.7-47.6 | | 10.0-18.4 | |
| Kit (mCi) | | | | | | | | | | | |
| Radioactive Concentration | | 7.47-8.99 | | 6.31-9.72 | | 7.61-8.81 | | 2.26-4.53 | | 0.95-1.77 | |
| Range Tested (mCi/mL) | | | | | | | | | | | |
| Number of [⁶⁸Ga]PSMA-11 | | 3 | | 3 | | 6 | | 3 | | 3 | |
| preparations* | | | | | | | | | | | |
| Gallium-68 Source | | GE Liquid | | GE Liquid | | 3 × GE | | 1 × EZAG | | 1 × EZAG | |
| | | target | | target | | Liquid Target | | GalliaPharm | | GalliaPharm | |
| | | | | | | 3 × EZAG | | 2 × IRE | | 2 × IRE | |
| | | | | | | GalliaPharm | | Galli Eo | | Galli Eo | |
| | Acceptance | | | | | | | | | | |
| Test | Criteria | T 0 | T 4 | T 0 | T 4 | T 0 | T 4 | T 0 | T 4 | T 0 | T 1 |
| Visual | Colourless to | C | C | C | C | C | C | C | C | C | C |
| Inspection | slightly yellow | | | | | | | | | | |
| | solution, free | | | | | | | | | | |
| | from visible | | | | | | | | | | |
| | particles | | | | | | | | | | |
| pH | 4.0-5.0 | C | C | C | C | C | C | C | C | C | C |

Radiochemical Purity (iTLC)

| | | | | | | | | | | | |
|---|---|---|---|---|---|---|---|---|---|---|---|
| Ga-68 | ≥95% | C | C | C | C | C | C | C | C | C | C |
| PSMA-11 | | 100 | 100 | 100 | 100 | 100 | 100 | 100 | 100 | 100 | 100 |
| (%) | | | | | | | | | | | |
| Free and | ≤5% | C | C | C | C | C | C | C | C | C | C |
| Colloidal | | 0 | 0 | 0 | 0 | 0 | 0 | 0 | 0 | 0 | 0 |
| Ga-68 (%) | | | | | | | | | | | |

Radiochemical Purity (HPLC)

| | | | | | | | | | | | |
|---|---|---|---|---|---|---|---|---|---|---|---|
| Ga-68 | ≥95% | C | C | C | C | C | C | C | C | C | C |
| PSMA-11 | | 100 | 100 | 100 | 100 | 100 | 100 | 100 | 100 | 100 | 100 |
| (%) | | | | | | | | | | | |

C = conforms to specifications. Average values for RCP for the runs are provided.
T 0 = Immediately after reconstitution and radiolabelling
T 1 = 1 hours after reconstitution and radiolabelling
T 4 = 4 hours after reconstitution and radiolabelling

In Table 1, the last two columns, the product was diluted to 10 mL by addition of 2.4 mL of 0.9% sodium chloride solution after the radiolabelling step. The data in Table 1 demonstrates that a range of 1-15 mg of ascorbic acid may be an effective radio-stabilizer for 10-76.9 mCi of [⁶⁸Ga]GaCl₃ starting activity, and a final product radioactive concentration of 0.95-9.72 mCi/mL. For activities <70 mCi, drug product stability may be maintained for up to 4 hours when lower amounts of ascorbic acid are added.

TABLE 2
Radiochemical purity data for batches of [⁶⁸Ga]PSMA-11 prepared with >70
mCi of [⁶⁸Ga]GaCl₃ with the inclusion of 10 mg, 20 mg and 50 mg of ascorbic acid

| Mass of Ascorbic Acid | | 10 mg | | 10 mg | | 20 mg | | 50 mg | |
|---|---|---|---|---|---|---|---|---|---|
| Included in the final | | | | | | | | | |
| radiolabelled product | | | | | | | | | |
| Activity Added to TLX-007 | | 458-500 | | 514-520 | | 514-544 | | 560 mCi | |
| Kit (mCi) | | | | | | | | | |
| Radioactive Concentration | | 43.4-47.3 | | 49.0-66.1 | | 48.3-51.9 | | 71.1 | |
| Range Tested (mCi/mL) | | | | | | | | | |
| Number of [⁶⁸Ga]PSMA-11 | | 3 | | 3 | | 2 | | 1 | |
| preparations* | | | | | | | | | |
| Gallium-68 Source | | GE Solid Target | | GE Solid Target | | GE Solid Target | | GE Solid Target | |
| | Acceptance | | | | | | | | |
| Test | Criteria | T 0 | T 4 | T 0 | T 6** | T 0 | T 6 | T 0 | T 6 |
| Visual | Colourless to | C | C | C | C | C | C | C | C |
| Inspection | slightly yellow | | | | | | | | |
| | solution, free | | | | | | | | |
| | from visible | | | | | | | | |
| | particles | | | | | | | | |
| pH | 4.0-5.0 | C | C | C | C | C | C | C | C |

Radiochemical Purity (iTLC)

| | | | | | | | | | |
|---|---|---|---|---|---|---|---|---|---|
| Ga-68 | ≥95% | C | C | C | C | C | C | C | C |
| PSMA-11 | | 100 | 100 | 100 | 100 | 97 | 98 | 99 | 100 |
| (%) | | | | | | | | | |
| Free and | ≤5% | C | C | C | C | C | C | C | C |
| Colloidal | | 0 | 0 | 0 | 0 | 3 | 2 | 1 | 0 |
| Ga-68 (%) | | | | | | | | | |

Radiochemical Purity (HPLC)

| | | | | | | | | | |
|---|---|---|---|---|---|---|---|---|---|
| Ga-68 | ≥95% | N/A | N/A | C | C | C | C | C | C |
| PSMA-11 | | | | 100 | 100 | 100 | 100 | 99 | 100 |
| (%) | | | | | | | | | |

C = conforms to specifications. Average values for RCP for the runs are provided.
T 0 = Immediately after reconstitution and radiolabelling
T 4 = 4 hours after reconstitution and radiolabelling
T 6 = 6 hours after reconstitution and radiolabelling
N/A = Data Not Available

In Table 2, in all cases the product was diluted to 10 mL by addition of 2.4 mL of 0.9% sodium chloride solution after the radiolabelling step. The data in Table 2 demonstrates that a range of 10-50 mg of ascorbic acid may be an effective radio-stabilizer for [⁶⁸Ga]PSMA-11 drug product for up to 6 hours, using 458-560 mCi of [⁶⁸Ga]GaCl₃ starting activity, and a final product radioactive concentration of 48.3-71.1 mCi/mL. For activities >70 mCi, drug product stability may be maintained for up to 6 hours when 10-50 mg of ascorbic acid are added. The range of ascorbic acid masses, from 1 mg-50 mg may be able to stabilise [⁶⁸Ga]PSMA-11 with varying amounts of [⁶⁸Ga]GaCl₃ starting activity and several sources of [⁶⁸Ga]GaCl₃. Stability the drug product was maintained across a range of starting radioactivity levels (10 mCi-560 mCi) and radioactive concentrations (0.95 mCi/mL-71.1 mCi/mL).

TABLE 3
Radiochemical purity data for batches of [⁶⁸Ga]PSMA-11 prepared with
<70 mCi of [⁶⁸Ga]GaCl₃ prepared with the EZAG GalliaPharm or IRE
Galli Eo generator or GE Liquid target with the inclusion of 25 mg of ascorbic acid.

| Mass of Sandoz Ascorbic Acid | | 25 mg | | 25 mg | | 25 mg | | 25 mg | |
|---|---|---|---|---|---|---|---|---|---|
| Included in the final | | | | | | | | | |
| radiolabelled product | | | | | | | | | |
| Activity Added to TLX-007 | | 46.0-46.1 | | 19.5-20.0 | | 23.3-24 | | 10.2 | |
| Kit (mCi) | | | | | | | | | |
| Radioactive Concentration | | 4.38-4.39 | | 1.85-1.90 | | 2.22-2.28 | | 0.97 | |
| Range Tested (mCi/mL) | | | | | | | | | |
| Number of [⁶⁸Ga]PSMA-11 | | 2 | | 2 | | 2 | | 1 | |
| preparations* | | | | | | | | | |
| Gallium-68 Source | | EZAG | | EZAG | | IRE Galli Eo | | IRE Galli Eo | |
| | | GalliaPharm | | GalliaPharm | | | | | |
| | Acceptance | | | | | | | | |
| Test | Criteria | T 0 | T 4 | T 0 | T 1 | T 0 | T 4 | T 0 | T 1 |
| Visual | Colourless to | C | C | C | C | C | C | C | C |
| Inspection | slightly yellow | | | | | | | | |
| | solution, free | | | | | | | | |
| | from visible | | | | | | | | |
| | particles | | | | | | | | |
| pH | 4.0-5.0 | C | C | C | C | C | C | C | C |

Radiochemical Purity (iTLC)

| | | | | | | | | | |
|---|---|---|---|---|---|---|---|---|---|
| Ga-68 | ≥95% | C | C | C | C | C | C | C | C |
| PSMA-11 | | 100 | 100 | 98 | 100 | 99 | 100 | 95 | 100 |
| (%) | | | | | | | | | |
| Free and | ≤5% | C | C | C | C | C | C | C | C |
| Colloidal | | 0 | 0 | 2 | 0 | 1 | 0 | 5 | 100 |
| Ga-68 (%) | | | | | | | | | |

Radiochemical Purity (HPLC)

| | | | | | | | | | |
|---|---|---|---|---|---|---|---|---|---|
| Ga-68 | ≥95% | C | C | C | C | C | C | C | C |
| PSMA-11 | | 100 | 100 | 100 | 100 | 100 | 100 | 100 | 100 |
| (%) | | | | | | | | | |

C = all results conform to specifications. Average values for RCP for the runs are provided.
T 0 = Immediately after reconstitution and radiolabelling
T 1 = 1 hour after reconstitution and radiolabelling
T 4 = 4 hours after reconstitution and radiolabelling

In Table 3, in all cases the product was diluted to 10 mL by addition of 2.4 mL of 0.9% sodium chloride solution after the radiolabelling step. The data in Table 3 shows that inclusion of 25 mg of ascorbic acid may stabilize the [⁶⁸Ga]PSMA-11 formulation for up to 4 hours.

TABLE 4
Radiochemical purity data for validation batches of [
⁶⁸Ga]PSMA-11 prepared with <70 mCi of [⁶⁸Ga]GaCl₃ prepared
with the EZAG GalliaPharm or IRE Galli Eo generator or GE
Liquid target with the inclusion of 25 mg of ascorbic acid.

| Mass of Sandoz Ascorbic Acid | | 25 mg | | 25 mg | | 25 mg | |
|---|---|---|---|---|---|---|---|
| Included in the final | | | | | | | |
| radiolabelled product | | | | | | | |
| Activity Added to TLX-007 | | 65.0-66.4 | | 51.6-62.7 | | 53.3-74.4 | |
| Kit (mCi) | | | | | | | |
| Radioactive Concentration | | 5.74-6.15 | | 4.56-5.88 | | 5.03-6.92 | |
| Range Tested (mCi/mL) | | | | | | | |
| Number of [⁶⁸Ga]PSMA-11 | | 3 | | 3 | | 3 | |
| preparations* | | | | | | | |
| Gallium-68 Source | | EZAG | | IRE | | GE Liquid | |
| | | GalliaPharm | | Galli Eo | | target | |
| | Acceptance | | | | | | |
| Test | Criteria | T 0 | T 4 | T 0 | T 4 | T 0 | T 4 |
| Visual | Colourless to | C | C | C | C | C | C |
| Inspection | slightly yellow | | | | | | |
| | solution, free | | | | | | |
| | from visible | | | | | | |
| | particles | | | | | | |
| pH | 4.0-5.0 | C | C | C | C | C | C |

Radiochemical Purity (iTLC)

| | | | | | | | |
|---|---|---|---|---|---|---|---|
| Ga-68 | ≥ 95% | C | C | C | C | C | C |
| PSMA-11 | | 100 | 98 | 100 | 99 | 99 | 100 |
| (%) | | | | | | | |
| Free and | ≤5% | C | C | C | C | C | C |
| Colloidal | | 0 | 2 | 0 | 1 | 1 | 0 |
| Ga-68 (%) | | | | | | | |

Radiochemical Purity (HPLC)

| | | | | | | | |
|---|---|---|---|---|---|---|---|
| Ga-68 | ≥95% | C | C | C | C | C | C |
| PSMA-11 | | 100 | 100 | 100 | 100 | 99 | 100 |
| (%) | | | | | | | |

C = all results conform to specifications. Average values for RCP for the runs are provided.
T 0 = Immediately after reconstitution and radiolabelling
T 4 = 4 hours after reconstitution and radiolabelling

In Table 4, in all cases the product was diluted to 10 mL by addition of 2.4 mL of 0.9% sodium chloride solution after the radiolabelling step. Summarised validation batch data for [⁶⁸Ga]GaCl₃ produced using EZAG GalliaPharm generator, IRE Galli Eo Generator and GE liquid target are shown in Table 4.

TABLE 5
Radiochemical purity data for validation batches of
[⁶⁸Ga]PSMA-11 prepared with >70 mCi [⁶⁸Ga]GaCl₃
from the GE Solid Target or the ARTMS Solid Target
with the inclusion of 25 mg of ascorbic acid.

| | | |
|---|---|---|
| Mass of Sandoz Ascorbic Acid | 25 mg | 25 mg |
| Included in the final | | |
| radiolabelled product | | |
| Activity Added to TLX-007 Kit | 537-551 | 555-564 |
| (mCi) | | |
| Radioactive Concentration | 50.2-51.6 | 51.1-52.7 |
| Range Tested (mCi/mL) | | |
| Number of [⁶⁸Ga]PSMA-11 | 3 | 3 |
| preparations* | | |
| Gallium-68 Source | GE Solid | ARTMS Solid |
| | Target | Target |

| | Acceptance | | | | |
|---|---|---|---|---|---|
| Test | Criteria | T0 | T6 | T0 | T6 |
| Visual | Colourless to | C | C | C | C |
| Inspection | slightly yellow | | | | |
| | solution, free | | | | |
| | from visible | | | | |
| | particles | | | | |
| pH | 4.0-5.0 | C | C | C | C |

Radiochemical Purity (iTLC)

| | | | | | |
|---|---|---|---|---|---|
| Ga-68 PSMA- | ≥95% | C | C | C | C |
| 11 | | 100% | 99% | 99% | 100% |
| Free and | ≤5% | C | C | C | C |
| Colloidal Ga- | | 0% | 1% | 1% | 0% |
| 68 | | | | | |

Radiochemical Purity (HPLC)

| | | | | | |
|---|---|---|---|---|---|
| Ga-68 PSMA- | ≥95% | C | C | C | C |
| 11 | | 100% | 100% | 99% | 100% |

C = all results conform to specifications. Average values for RCP for the runs are provided.
T0 = Immediately after reconstitution and radiolabelling
T6 = 6 hours after reconstitution and radiolabelling

In Table 5, in all cases the product was diluted to 10 mL by addition of 2.4 mL of 0.9% sodium chloride solution after the radiolabelling step. Summarised validation batch data for [⁶⁸Ga]GaCl₃ produced using GE solid target and ARTMS solid target are shown in Table 5.

### Control Experiment, (No Ascorbic Acid)

TABLE 6
control experiment using 70 mCi [⁶⁸Ga]GaCl₃
from the GE Solid Target and no ascorbic acid.

| | |
|---|---|
| Mass of Sandoz Ascorbic Acid | 0 mg |
| Included in the final | |
| radiolabelled product | |
| Activity Added to TLX-007 Kit | 70 |
| (mCi) | |
| Number of [⁶⁸Ga]PSMA-11 | 1 |
| preparations* | |
| Gallium-68 Source | GE Solid Target |

| | Acceptance | | |
|---|---|---|---|
| Test | Criteria | T0 | T4 |
| Visual | Colourless to | C | C |
| Inspection | slightly yellow | | |
| | solution, free | | |
| | from visible | | |
| | particles | | |
| pH | 4.0-5.0 | C | C |

Radiochemical Purity (iTLC)

| | | | |
|---|---|---|---|
| Ga-68 PSMA- | ≥95% | C | C |
| 11 | | 97% | 95% |

Radiochemical Purity (HPLC)

| | | | |
|---|---|---|---|
| Ga-68 PSMA- | ≥95% | X | X |
| 11 | | 87% | 65% |

C = all results conform to specifications.
X = fail, obtained results falls outside specifications.
T0 = Immediately after reconstitution and radiolabelling
T4 = 4 hours after reconstitution and radiolabelling

### Example 5 Ascorbic Acid Bracketing Matrix

The kits of Example 1, Example 2 or Example 3, with amounts of ascorbic acid as set out in Table 7 were used in the experiments for the ascorbic acid bracketing matrix. Three strengths of [⁶⁸Ga]Ga-PSMA-11 will be tested, i.e. 50 mCi/ml, 7 mCi/ml and 1 mCi/ml. Radiochemical purity was determined at different time points (TO, T1, T4 or T6), as set out in Table 7. The results re provided in Table 8, Table 9 and Table 10.

TABLE 7
the ascorbic acid bracketing matrix

| | | Strength (mCi/mL) | | | | | | | | |
|---|---|---|---|---|---|---|---|---|---|---|
| | | 50 | | | 7 | | | 1 | | |
| | | Ascorbic Acid | | | | | | | | |
| | | concentration (mg) | | | | | | | | |
| | | 20 | 25 | 31.25 | 20 | 25 | 31.25 | 20 | 25 | 31.25 |
| Source of | GE Solid Target | T 0, T 6 | T 0, T 6 | T 0, T 6 | NT | NT | NT | NT | NT | NT |
| [⁶⁸Ga] | GE Liquid Target | N/A | N/A | N/A | NT | T 0, T 4 | NT | NT | NT | NT |
| GaCl₃ | EZAG | N/A | N/A | N/A | NT | T 0, T 4 | T 0, T 4 | NT | NT | T 0, T 1 |
| | GalliaPharm100 | | | | | | | | | |
| | mCi Generator | | | | | | | | | |
| | IRE Galli Eo 100 | N/A | N/A | N/A | T 0, T 4 | T 0, T 4 | NT | T 0, T 1 | T 0, T 1 | NT |
| | mCi Generator | | | | | | | | | |

Wherein:
N/A: Not Applicable. This activity concentration is not achievable with this source of [⁶⁸Ga]GaCl₃.
NT: Not tested.
T0: Immediately after reconstitution and radiolabelling.
T1:1 hour after reconstitution and radiolabelling.
T4:4 hours after reconstitution and radiolabelling.
T6:6 hours after reconstitution and radiolabelling.

The data in Table 8 confirms that stability of the [⁶⁸Ga]PSMA-11 is maintained for up to 6 hours when the lowest amount of ascorbic acid (20 mg) is used in the highest strength of the product (48.3-51.9 mCi/mL), representing the “worst case” scenario for the product (highest strength, lowest mass of stabilizer).

The data in Tables 9 and Table 10 correspond to the strengths of the product when prepared with “high activity” generators near their calibration date (5.1-5.88 mCi/mL), and simulate the use of generators near expiry (0.84-2.54 mCi/mL), respectively. The data confirms that that stability of the [⁶⁸Ga]PSMA-11 is maintained when 18.75-31.25 mg of ascorbic acid is included as a radiostabilizer. From the above data, a range of 25 mg±20% (corresponding to the “worst case” scenario described above) maintains the stability of [⁶⁸Ga]PSMA-11, demonstrating that the radiolabelling procedure according to the invention are robust.

TABLE 8
Ascorbic Acid Mass Bracketing when Radiolabelling at a strength of ~50 mCi/mL
Ascorbic Acid Mass Bracketing for ~50 mCi/mL

| Mass of Sandoz Ascorbic Acid | | 20 mg | | 25 mg | | 31.25 mg | |
|---|---|---|---|---|---|---|---|
| Included in the final | | | | | | | |
| radiolabelled product | | | | | | | |
| Activity Added to TLX-007 | | 514-544 | | 537-551 | | 500-544 | |
| Kit (mCi) | | | | | | | |
| Radioactive Concentration | | 48.3-51.9 | | 50.2-51.6 | | 48.1-51.7 | |
| Range Tested (mCi/mL) | | | | | | | |
| Number of [⁶⁸Ga]PSMA-11 | | 2 | | 3 | | 2 | |
| preparations* | | | | | | | |
| Gallium-68 Source | | GE Solid Target | | GE Solid Target | | GE Solid Target | |
| | Acceptance | | | | | | |
| Test | Criteria | T 0 | T 6 | T 0 | T 6 | T 0 | T 6 |
| Visual | Colourless to | C | C | C | C | C | C |
| Inspection | slightly yellow | | | | | | |
| | solution, free | | | | | | |
| | from visible | | | | | | |
| | particles | | | | | | |
| pH | 4.0-5.0 | C | C | C | C | C | C |

Radiochemical Purity (iTLC)**

| | | | | | | | |
|---|---|---|---|---|---|---|---|
| Ga-68 | ≥95% | 97 | 98 | 100 | 99 | 99 | 99 |
| PSMA-11 | | | | | | | |
| (%) | | | | | | | |
| Free and | ≤5% | 3 | 2 | 0 | 1 | 1 | 1 |
| Colloidal | | | | | | | |
| Ga-68 (%) | | | | | | | |

Radiochemical Purity (HPLC)**

| | | | | | | | |
|---|---|---|---|---|---|---|---|
| Ga-68 | ≥95% | 100 | 100 | 100 | 100 | 100 | 100 |
| PSMA-11 | | | | | | | |
| (%) | | | | | | | |

**Average values for RCP for the runs are provided.
C = Conforms to specifications.
T 0 = Immediately after reconstitution and radiolabelling
T 6 = 6 hours after reconstitution and radiolabelling

TABLE 9
Ascorbic Acid Mass Bracketing when Radiolabelling at a strength of ~7 mCi/mL
Ascorbic Acid Mass Bracketing for ~7 mCi/mL

| Mass of Sandoz Ascorbic | | 18.75 mg | | 25 mg | | 31.25 mg | |
|---|---|---|---|---|---|---|---|
| Acid Included in the final | | | | | | | |
| radiolabelled product | | | | | | | |
| Activity Added to TLX-007 | | 51.0-58.0 | | 51.6-74.4 | | 61.5-62.0 | |
| Kit (mCi) | | | | | | | |
| Radioactive Concentration | | 5.55-6.5 | | 4.56-6.92 | | 5.85-5.88 | |
| Range Tested (mCi/mL) | | | | | | | |
| Number of [⁶⁸Ga]PSMA-11 | | 2 | | 9 | | 2 | |
| preparations* | | | | | | | |
| Gallium-68 Source | | IRE Galli Eo | | 3 × GE Liquid Target | | EZAG | |
| | | | | 3 × IRE Galli Eo | | GalliaPharm | |
| | | | | 3 × EZAG | | | |
| | Acceptance | | | GalliaPharm | | | |
| Test | Criteria | T 0 | T 4 | T 0 | T 4 | T 0 | T4 |
| Visual | Colourless to | C | C | C | C | C | C |
| Inspection | slightly yellow | | | | | | |
| | solution, free | | | | | | |
| | from visible | | | | | | |
| | particles | | | | | | |
| pH | 4.0-5.0 | C | C | C | C | C | C |

Radiochemical Purity (iTLC)**

| | | | | | | | |
|---|---|---|---|---|---|---|---|
| Ga-68 | ≥95% | 100 | 100 | 100 | 99 | 100 | 100 |
| PSMA-11 | | | | | | | |
| (%) | | | | | | | |
| Free and | ≤5% | 0 | 0 | 0 | 1 | 0 | 0 |
| Colloidal | | | | | | | |
| Ga-68 (%) | | | | | | | |

Radiochemical Purity (HPLC)**

| | | | | | | | |
|---|---|---|---|---|---|---|---|
| Ga-68 | ≥95% | 100 | 100 | 100 | 100 | 100 | 100 |
| PSMA-11 | | | | | | | |
| (%) | | | | | | | |

**Average values for RCP for the runs are provided
C = Conforms to specifications
T 0 = Immediately after reconstitution and radiolabelling
T 4 = 4 hours after reconstitution and radiolabelling

TABLE 10
Ascorbic Acid Mass Bracketing when Radiolabelling at a strength of ~1 mCi/mL
Ascorbic Acid Mass Bracketing for ~1 mCi/mL

| Mass of Sandoz Ascorbic Acid | | 18.75 mg | | 25 mg | | 31.25 mg | |
|---|---|---|---|---|---|---|---|
| Included in the final | | | | | | | |
| radiolabelled product | | | | | | | |
| Activity Added to TLX-007 | | 8.9-11.5 | | 8.8-12.7 | | 15.2-26.9 | |
| Kit (mCi) | | | | | | | |
| Radioactive Concentration | | 0.85-1.09 | | 0.84-1.21 | | 1.44-2.54 | |
| Range Tested (mCi/mL) | | | | | | | |
| Number of [⁶⁸Ga]PSMA-11 | | 2 | | 2 | | 2 | |
| preparations* | | | | | | | |
| Gallium-68 Source | | IRE Galli Eo | | 2 × IRE Galli Eo | | EZAG GalliaPharm | |
| | Acceptance | | | | | | |
| Test | Criteria | T 0 | T 1 | T 0 | T 1 | T 0 | T 1 |
| Visual | Colourless to | C | C | C | C | C | C |
| Inspection | slightly yellow | | | | | | |
| | solution, free | | | | | | |
| | from visible | | | | | | |
| | particles | | | | | | |
| pH | 4.0-5.0 | C | C | C | C | C | C |

Radiochemical Purity (iTLC)**

| | | | | | | | |
|---|---|---|---|---|---|---|---|
| Ga-68 | ≥95% | 100 | 100 | 100 | 100 | 100 | 100 |
| PSMA-11 | | | | | | | |
| (%) | | | | | | | |
| Free and | ≤5% | 0 | 0 | 0 | 0 | 0 | 0 |
| Colloidal | | | | | | | |
| Ga-68 (%) | | | | | | | |

Radiochemical Purity (HPLC)**

| | | | | | | | |
|---|---|---|---|---|---|---|---|
| Ga-68 | ≥95% | 100 | 100 | 99 | 100 | 100 | 100 |
| PSMA-11 | | | | | | | |
| (%) | | | | | | | |

**Average values for RCP for the runs are provided
C = Conforms to specifications
T 0 = Immediately after reconstitution and radiolabelling
T 1 = 1 hour after reconstitution and radiolabelling

1. A method for radiolabelling a chelate-functionalized targeting agent with a metal radionuclide being gallium-68 or gallium-67, comprising the steps of:
a) providing a stabiliser that prevents radiolysis (product degradation) of the chelate-functionalized targeting agent, wherein said stabiliser is selected from the group consisting of: ascorbic acid, dehydroascorbic acid, gentisic acid, cysteine and methionine, sodium ascorbate, or a salt thereof, preferably as a solution to the radiolabelling mixture prior to radiolabelling;
b) providing a chelate-functionalized targeting agent, able to chelate the radioactive metal in the radiolabelling conditions;
c) combining the mixture of a) and c); and,
d) adding a radioactive metal to the mixture obtained in c), thereby radiolabelling the chelate-functionalized targeting agent with gallium-68 or gallium-67;
wherein the method optionally further comprises mixing the stabiliser of a) with a buffering agent or buffer solution, allowing to maintain the pH in the range 3 to 8; and/or
wherein the method optionally further comprises adding a metal inhibitor to said targeting agent of b), said metal inhibitor being a co-chelating agent, capable of inactivating metals other than radioactive metal without interfering with the chelation between the radioactive metal and the said chelate-functionalized targeting agent, under the conditions of the labelling reaction.

2. The method according to claim 1, for radiolabelling a chelate-functionalized targeting agent with a metal radionuclide thereby producing a radiolabelled chelate-functionalized targeting agent with an activity of at least 50.0 mCi.

3. The method according to claim 1, wherein the radioactive metal is provided as a solution with an radioactive concentration of at least 5.0 mCi/ml.

4. The method according to claim 1, wherein the mixture obtained in step c) comprises at least 10.0 mg.

5. The method according to claim 1, wherein the mixture obtained in step c) comprises at most 100.0 mg.

6. The method according to claim 1, wherein the mixture obtained in step c) comprises at least 1.0 mg of stabiliser per 50 mCi metal radionuclide in step f).

7. The method according to claim 1, wherein the mixture obtained in step c) comprises at most 100.0 mg of stabiliser per 50 mCi metal radionuclide in step f).

8. The method according to claim 1, wherein the amount of stabiliser in the mixture obtained in step c), is so that the weight ratio of the weight of stabiliser over the weight of the chelate-functionalized targeting agent in the mixture obtained in step c), is at least 40.

9. The method according to claim 1, wherein the solution of stabiliser has a concentration of at least 0.14 mg/ml; wherein the stabilizer is ascorbic acid or a salt thereof.

10. The method according to claim 1, wherein the stabiliser is ascorbic acid, dehydroascorbic acid or a salt thereof.

11. The method according to claim 1, wherein the method further comprises providing at least 3.0 patient doses.

12. The method according to claim 1, wherein said targeting agent and metal inhibitor are present in a buffer allowing to maintain the pH in the range 3 to 8.

13. The method according to claim 1, wherein said targeting agent and metal inhibitor are present in a buffer selected from the group consisting of: phosphate, nitrate, HEPES, acetate, formate, TRIS, and citrate or a mixture thereof, preferably in an acetate buffer, more preferably a sodium acetate buffer.

14. The method according to claim 1, wherein the chelate functional group of the targeting agent is HBED or derivatives thereof such as HBED-CC.

15. The method according to claim 1, wherein said metal inhibitor is a sugar, preferably a short-chain sugar or oligosaccharide, such as comprising up to 7 monosaccharide units.

16. The method according to claim 1, wherein said metal inhibitor is selected from the group comprising: monosaccharides and their derivatives, disaccharides and their derivatives, trisaccharides and their derivatives, tetrasaccharides and their derivatives, and cyclic oligosaccharides and their derivatives.

17. The method according to claim 1, wherein said metal inhibitor is selected from the group comprising: Glucose, D-Fructose, Beta-cyclodextrin, and D-Mannose, more preferably D-mannose.

18. The method according to claim 1, wherein said metal inhibitor and said functionalised agent are not chemically linked.

19. The method according to claim 1, wherein said metal inhibitor and said functionalised agent are chemically linked, through a linker that is unstable in the radiolabelling conditions.

20. The method according to claim 1, wherein said chelate-functionalized targeting agent is Glu-urea-Lys-HBED-CC (gozetotide or PSMA-11).

21. The method according to claim 1, wherein the radiolabelling reaction is carried out at ambient or room temperature.

22. The method according to claim 1, wherein the radiolabelling is performed at a pH comprised between 3 and 8, preferably between 3.5 and 7.5, more preferably between 3.5 and 7.

23. A radiolabelled chelate-functionalized targeting agent obtained by the method according claim 1.

24. The radiolabelled chelate-functionalized targeting agent according to claim 23, wherein the radiolabelled chelate-functionalized targeting agent comprises gallium-68 radiolabelled Glu-urea-Lys-HBED-CC (gozetotide or PSMA-11).

25. A radiolabelling kit for producing a radiolabelled chelate-functionalized targeting agent with an activity of at least 50.0 mCi, comprising:
(a) a chelate-functionalized targeting agent, able to chelate the radioactive metal in the radiolabelling conditions;
(b) a stabiliser selected from the group consisting of: ascorbic acid, sodium ascorbate, dehydroascorbic acid, gentisic acid, cysteine and methionine, or a salt thereof, preferably as a solution; and
(c) gallium-68 as radioactive metal; and, optionally one or more of:
a metal inhibitor, which is a co-chelating agent, capable of inactivating metals other than radioactive metal without interfering with the chelation between the radioactive metal and the said chelate-functionalized targeting agent, under the conditions of the labelling reaction; and/or
a buffering agent or buffer solution, allowing to maintain the pH in the range 3 to 8.

26. A method of detecting a prostate tumour or cancer, comprising the steps of:
1) radiolabelling PSMA-11 (gozetotide) with gallium-68 according to the method claim 1,
2) administering to a subject a diagnostic amount of gallium-68 radiolabelled PSMA-11 (gozetotide); and,
3) detecting binding of said gallium-68 radiolabelled PSMA-11 (gozetotide) using PET or PET/CT imaging methods.

27. The method according to claim 26, wherein said detection is used for:
(i) initial staging of prostate cancer into intermediate, unfavourable, high, or very high risk prostate cancer,
(ii) detecting suspected recurrence of prostate cancer and/or detection of metastasis,
(iii) selection for radiotherapeutic treatment such as with Lutetium (177Lu) vipivotide tetraxetan (Pluvicto)
(iv) monitoring prostate cancer for progression into Non-Metastatic or Metastatic Castration-Resistant Prostate Cancer (nmCRPC or mCRPC), or
(v) determining response to (radio) therapy.

28. The method according to claim 26, wherein said detection method is used to replace the need for taking a prostate biopsy or is used in PET or PET/CT scan with MRI in clinically significant or intermediate favourable prostate cancers, or in MRI for active surveillance of prostate cancer.
